(19) United States
(12) Patent Application Publication
(10) Pub. No.: US 2007/0259361 A1
(43) Pub. Date: Nov. 8, 2007

(54) METHODS, COMPOSITIONS, AND KITS FOR THE DETECTION AND MONITORING OF BLADDER CANCER

(75) Inventors: Paul A. Algate, Issaquah, WA (US); Jane Mannion, Newbury Park, CA (US); Brian Gordon, Issaquah, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Corixa Corporation, Hamilton, MT (US)

(21) Appl. No.: 11/734,154

(22) Filed: Apr. 11, 2007

Related U.S. Application Data
(60) Provisional application No. 60/791,027, filed on Apr. 11, 2006.

(51) Int. Cl. C12Q 1/68; C07H 21/04

(52) U.S. Cl. 435/6; 536/24.3

(57) ABSTRACT

Methods and compositions for the diagnosis and monitoring of bladder cancer are disclosed.

### CROSS-REFERENCE TO RELATED APPLICATION

This application claims the benefit under 35 U.S.C. § 119(e) of U.S. Provisional Patent Application No. 60/791,027 filed Apr. 11, 2006; where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 210121_{—}615_SEQUENCE_LISTING.txt. The text file is 28 KB, was created on Apr. 11, 2007, and is being submitted electronically via EFS-Web, concurrent with the filing of the specification.

### BACKGROUND OF THE INVENTION

1. Field of the Invention

The present invention relates generally to the field of cancer diagnostics. More specifically, the present invention relates to methods, compositions, and kits for use in detecting the expression of cancer-associated polynucleotides and polypeptides in a biological sample.

2. Description of the Related Art

Cancer remains one of the most significant health problems throughout the world. Although advances have been made in the detection, diagnosis and treatment of cancer, the development of improved techniques for the early and accurate detection of cancer has the potential to offer clinicians a broader array of information and treatment options in their efforts to combat the disease.

In the United States, bladder cancer is the 4th most common cancer in men and the 9th most common cancer in women. Overall, it is the 6th most common cancer with over 50,000 cases per year. The American Cancer Society estimates that 63,210 new cases of bladder cancer will be found in the United States during 2005. Over 12,000 deaths are expected to result from this malignancy each year. The five-year survival rate is good when bladder cancer is found and treated early, but drops to 50% with local metastasis and 6% if distant organs are involved. (American Cancer Society, www dot cancer dot org).

Molecular assays, particularly those using nucleic acid amplification techniques, can greatly improve the diagnostic sensitivity for detecting malignant cells. Despite these advances, molecular diagnostic approaches remain hampered by the relative paucity of effective and complementary cancer-specific markers. Thus, there remains a need for diagnostic approaches having improved sensitivity, specificity, tumor coverage, and correlation to disease state. The present invention achieves these and other related objectives.

### SUMMARY OF THE INVENTION

One aspect of the present invention provides compositions for detecting bladder cancer cells in a biological sample comprising an oligonucleotide specific for any one of the cancer-associated polynucleotides recited in SEQ ID NOs: 1-2, 4-5, 7-8, 10-12, 15-16 and 18, or the complement thereof. In certain embodiments, an oligonucleotide of the present invention is specific for a polynucleotide encoding any one of the cancer-associated polypeptides recited in SEQ ID NOs: 3, 6, 9, 13-14 and 17, or the complement thereof.

Another aspect of the invention provides compositions for detecting bladder cancer cells in a biological sample comprising at least two oligonucleotide primers specific for any one of the cancer-associated polynucleotides recited in SEQ ID NOs: 1-2, 4-5, 7-8, 10-12, 15-16 and 18, or the complement thereof. In certain embodiments, the compositions for detecting bladder cancer cells in a biological sample comprise at least two oligonucleotide primers specific for a polynucleotide encoding any one of the cancer-associated polypeptides recited in SEQ ID NOs: 3, 6, 9, 13-14 and 17, or the complement thereof. In this regard, the two oligonucleotides may hybridize to the same strand or to opposite strands of the polynucleotide of interest.

A further aspect of the invention provides compositions for detecting bladder cancer cells in a biological sample comprising at least two of a first oligonucleotide primer pair specific for any one of the polynucleotides recited in SEQ ID NOs: 1-2, 4-5, 7-8, 10-12, 15-16 and 18, or the complement thereof; a second oligonucleotide primer pair specific for any one of the polynucleotides recited in SEQ ID NOs: 1-2, 4-5, 7-8, 10-12, 15-16 and 18, or the complement thereof; a third oligonucleotide primer pair specific for any one of the polynucleotides recited in SEQ ID NOs: 1-2, 4-5, 7-8, 10-12, 15-16 and 18, or the complement thereof; a fourth oligonucleotide primer pair specific for any one of the polynucleotides recited in SEQ ID NOs: 1-2, 4-5, 7-8, 10-12, 15-16 and 18, or the complement thereof; a fifth oligonucleotide primer pair specific for any one of the polynucleotides recited in SEQ ID NOs: 1-2, 4-5, 7-8, 10-12, 15-16 and 18, or the complement thereof; and, a sixth oligonucleotide primer pair specific for any one of the polynucleotides recited in SEQ ID NOs: 1-2, 4-5, 7-8, 10-12, 15-16 and 18, or the complement thereof; wherein the first, second, third, fourth, fifth and sixth primer pairs are specific for different polynucleotides from among the polynucleotides recited in SEQ ID NOs: 1-2, 4-5, 7-8, 10-12, 15-16 and 18. As noted elsewhere herein, a primer pair generally comprises a first primer and a second primer where the first and second primers specifically hybridize to opposite strands of a target polynucleotide.

Yet a further aspect of the invention provides compositions for detecting bladder cancer cells in a biological sample comprising any one or more of the polypeptide sequences recited in SEQ ID NOs: 3, 6, 9, 13-14 and 17, or a polypeptide sequence encoded by a polynucleotide sequence set forth in any one of SEQ ID NOs: 1-2, 4-5, 7-8, 10-12, 15-16 and 18, or a fragment of any of said polypeptide sequences wherein said fragment is useful in the detection of bladder cancer cells. In certain embodiments, the compositions comprise at least two, three, four, five or more of the polypeptide sequences recited in SEQ ID NOs: 3, 6, 9, 13-14 and 17 or the polypeptide sequences encoded by the polynucleotides recited in SEQ ID NOs: 1-2, 4-5, 7-8, 10-12, 15-16 and 18.

An additional aspect of the invention provides compositions for detecting bladder cancer cells in a biological sample comprising an antibody that specifically recognizes any one of the polypeptide sequences recited in SEQ ID NOs: 3, 6, 9, 13-14 and 17 or the polypeptide sequences encoded by the polynucleotides recited in SEQ ID NOs: 1-2, 4-5, 7-8, 10-12, 15-16 and 18. In certain embodiments, the compositions comprise at least two, three, four, five or more antibodies that each specifically recognize any one of the polypeptide sequences recited in SEQ ID NOs: 3, 6, 9, 13-14 and 17 or the polypeptide sequences encoded by the polynucleotides recited in SEQ ID NOs: 1-2, 4-5, 7-8, 10-12, 15-16 and 18.

In another aspect of the invention, diagnostic kits are provided for detecting bladder cancer cells in a biological sample comprising at least one oligonucleotide primer or probe, wherein the oligonucleotide primer or probe is specific for any one of the cancer-associated polynucleotides recited in SEQ ID NO: 1-2, 4-5, 7-8, 10-12, 15-16 and 18, or the complement thereof or a polynucleotide encoding any one of the amino acid sequences set forth in SEQ ID NOs:3, 6, 9, 13-14 and 17, or the complement thereof.

A further aspect of the invention provides diagnostic kits for detecting bladder cancer cells in a biological sample comprising at least two oligonucleotide primers specific for any one of the cancer-associated polynucleotides recited in SEQ ID NO: 1-2, 4-5, 7-8, 10-12, 15-16 and 18, or the complement thereof or a polynucleotide encoding any one of the amino acid sequences set forth in SEQ ID NOs:3, 6, 9, 13-14 and 17, or the complement thereof.

Another aspect of the invention provides diagnostic kits for detecting bladder cancer cells in a biological sample comprising at least two of a first oligonucleotide primer pair specific for any one of the polynucleotides recited in SEQ ID NOs: 1-2, 4-5, 7-8, 10-12, 15-16 and 18, or the complement thereof or a polynucleotide encoding any one of the amino acid sequences set forth in SEQ ID NOs:3, 6, 9, 13-14 and 17, or the complement thereof; a second oligonucleotide primer pair specific for any one of the polynucleotides recited in SEQ ID NOs: 1-2, 4-5, 7-8, 10-12, 15-16 and 18, or the complement thereof or a polynucleotide encoding any one of the amino acid sequences set forth in SEQ ID NOs:3, 6, 9, 13-14 and 17, or the complement thereof; a third oligonucleotide primer pair specific for any one of the polynucleotides recited in SEQ ID NOs: 1-2, 4-5, 7-8, 10-12, 15-16 and 18, or the complement thereof or a polynucleotide encoding any one of the amino acid sequences set forth in SEQ ID NOs:3, 6, 9, 13-14 and 17, or the complement thereof; a fourth oligonucleotide primer pair specific for any one of the polynucleotides recited in SEQ ID NOs: 1-2, 4-5, 7-8, 10-12, 15-16 and 18, or the complement thereof or a polynucleotide encoding any one of the amino acid sequences set forth in SEQ ID NOs:3, 6, 9, 13-14 and 17, or the complement thereof; a fifth oligonucleotide primer pair specific for any one of the polynucleotides recited in SEQ ID NOs: 1-2, 4-5, 7-8, 10-12, 15-16 and 18, or the complement thereof or a polynucleotide encoding any one of the amino acid sequences set forth in SEQ ID NOs:3, 6, 9, 13-14 and 17, or the complement thereof; and, a sixth oligonucleotide primer pair specific for any one of the polynucleotides recited in SEQ ID NOs: 1-2, 4-5, 7-8, 10-12, 15-16 and 18, or the complement thereof or a polynucleotide encoding any one of the amino acid sequences set forth in SEQ ID NOs:3, 6, 9, 13-14 and 17, or the complement thereof; wherein the first, second, third, fourth, fifth and sixth primer pairs are specific for different polynucleotides from among the polynucleotides recited in SEQ ID NOs: 1-2, 4-5, 7-8, 10-12, 15-16 and 18.

An additional aspect of the invention provides diagnostic kits for detecting antibodies specific for a cancer-associated marker in a biological sample comprising at least one cancer-associated polypeptide recited in any one of SEQ ID NOs: 3, 6, 9, 13-14 and 17, or a polypeptide sequence encoded by a polynucleotide sequence set forth in any one of SEQ ID NOs: 1-2, 4-5, 7-8, 10-12, 15-16 and 18, or a fragment of any of said polypeptide sequences wherein said fragment is specifically recognized by antibodies specific for the corresponding full-length polypeptide.

Another aspect of the invention provides diagnostic kits for detecting bladder cancer cells in a biological sample comprising at least one isolated antibody, or antigen-binding fragment thereof, that specifically binds to any one of the cancer-associated polypeptides recited in SEQ ID NOs: 3, 6, 9, 13-14 and 17, or a polypeptide sequence encoded by a polynucleotide sequence set forth in any one of SEQ ID NOs: 1-2, 4-5, 7-8, 10-12, 15-16 and 18.

Further aspects of the present invention provide for arrays. In one particular aspect, the invention provides arrays for detecting bladder cancer cells in a biological sample comprising at least one oligonucleotide primer or probe, wherein the oligonucleotide primer or probe is specific for any one of the cancer-associated polynucleotides recited in SEQ ID NOs: 1-2, 4-5, 7-8, 10-12, 15-16 and 18, or the complement thereof or a polynucleotide encoding any one of the amino acid sequences set forth in SEQ ID NOs:3, 6, 9, 13-14 and 17, or the complement thereof. In one embodiment, a first oligonucleotide is specific for the nucleic acid sequence set forth in SEQ ID NO:1 or 2 or a nucleic acid sequence encoding an amino acid sequence set forth in SEQ ID NO:3, a second oligonucleotide is specific for the nucleic acid sequence set forth in SEQ ID NO:4 or 5 or a nucleic acid sequence encoding an amino acid sequence set forth in SEQ ID NO:6, a third oligonucleotide is specific for the nucleic acid sequence set forth in SEQ ID NO:7 or 8 or a nucleic acid sequence encoding an amino acid sequence set forth in SEQ ID NO:9, a fourth oligonucleotide is specific for the nucleic acid sequence set forth in SEQ ID NO:10, 11 or 12 or a nucleic acid sequence encoding an amino acid sequence set forth in SEQ ID NO: 13 or 14, a fifth oligonucleotide is specific for the nucleic acid sequence set forth in SEQ ID NO:15 or 16 or a nucleic acid sequence encoding an amino acid sequence set forth in SEQ ID NO:17, and a sixth oligonucleotide is specific for the nucleic acid sequence set forth in SEQ ID NO:18.

A further aspect of the invention provides arrays for detecting antibodies specific for a cancer-associated marker in a biological sample comprising at least one cancer-associated polypeptide recited in any one of SEQ ID NOs: 3, 6, 9, 13-14 and 17, or a polypeptide sequence encoded by a polynucleotide sequence set forth in any one of SEQ ID NOs: 1-2, 4-5, 7-8, 10-12, 15-16 and 18, or a fragment of any of said polypeptide sequences wherein said fragment is specifically recognized by antibodies specific for the corresponding full-length polypeptide. In one embodiment, a first cancer-associated marker comprises the amino acid sequence set forth in SEQ ID NO:3, a second cancer-associated marker comprises the amino acid sequence set forth in SEQ ID NO:6, a third cancer-associated marker comprises the amino acid sequence set forth in SEQ ID NO:9, a fourth cancer-associated marker comprises the amino acid sequence set forth in SEQ ID NO:13 or 14, a fifth cancer-associated marker comprises the amino acid sequence set forth in SEQ ID NO: 17, and a sixth cancer-associated marker comprises the amino acid sequence encoded by the polynucleotide set forth in SEQ ID NO:18.

Yet an additional aspect of the invention provides arrays for detecting bladder cancer cells in a biological sample comprising at least one isolated antibody, or antigen-binding fragment thereof, that specifically binds to any one of the cancer-associated polypeptides recited in SEQ ID NOs: 3, 6, 9, 13-14 and 17 or a polypeptide sequence encoded by a polynucleotide sequence set forth in any one of SEQ ID NOs: 1-2, 4-5, 7-8, 10-12, 15-16 and 18. In one embodiment, a first antibody is specific for the amino acid sequence set forth in SEQ ID NO:3, a second antibody is specific for the amino acid sequence set forth in SEQ ID NO:6, a third antibody is specific for the amino acid sequence set forth in SEQ ID NO: 9, a fourth antibody is specific for the amino acid sequence set forth in SEQ ID NO:13 or 14, a fifth antibody is specific for the amino acid sequence set forth in SEQ ID NO:17, and a sixth antibody is specific for the amino acid sequence encoded by the polynucleotide set forth in SEQ ID NO:18.

According to one aspect of the invention, methods are provided for detecting the presence of cancer cells in a biological sample comprising the steps of: detecting the level of expression in the biological sample of at least one cancer-associated marker, wherein the cancer-associated marker comprises a polynucleotide set forth in any one of SEQ ID NOs: 1-2, 4-5, 7-8, 10-12, 15-16 and 18 or a polypeptide set forth in any one of SEQ ID NOs: 3, 6, 9, 13-14 and 17 or a polypeptide sequence encoded by a polynucleotide sequence set forth in any one of SEQ ID NOs: 1-2, 4-5, 7-8, 10-12, 15-16 and 18 and, comparing the level of expression detected in the biological sample for the cancer-associated marker to a predetermined cut-off value for the cancer-associated marker; wherein a detected level of expression above the predetermined cut-off value for the cancer-associated marker is indicative of the presence of cancer cells in the biological sample.

The cancer to be detected according to the methods of the invention may be any cancer type that expresses one or more of the cancer-associated markers described herein. In certain illustrative embodiments, the cancer is a bladder cancer.

The biological sample to be tested according to the methods of the invention may be any type of biological sample suspected of containing cancer-associated markers, antibodies to such cancer-associated markers and/or cancer cells expressing such markers or antibodies. In one embodiment, for example, the biological sample is a tissue sample suspected of containing cancer cells. In other embodiments, the biological sample is selected from the group consisting of a biopsy sample, lavage sample, sputum sample, serum sample, peripheral blood sample, lymph node sample, bone marrow sample, urine sample, and pleural effusion sample.

In certain embodiments of the invention, the step of detecting expression of a cancer-associated marker comprises detecting mRNA expression of a cancer-associated marker, for example, using a nucleic acid hybridization technique or a nucleic acid amplification method. Such methods for detecting mRNA expression are well-known and established in the art and may include, but are not limited to, transcription-mediated amplification (TMA), polymerase chain reaction amplification (PCR), reverse-transcription polymerase chain reaction amplification (RT-PCR), ligase chain reaction amplification (LCR), strand displacement amplification (SDA), and nucleic acid sequence based amplification (NASBA), as further described herein. In certain embodiments, the cancer-associated marker comprises a nucleic acid sequence set forth in any one of SEQ ID NOs: 1-2, 4-5, 7-8, 10-12, 15-16 and 18 or a polynucleotide encoding any one of the amino acid sequences set forth in SEQ ID NOs:3, 6, 9, 13-14 and 17.

In certain other embodiments of the invention, the step of detecting expression of a cancer-associated marker comprises detecting protein expression of a cancer-associated marker. Methods for detecting protein expression may include any of a variety of well-known and established techniques. For example, in certain embodiments, the step of detecting protein expression comprises detecting protein expression using an immunoassay, such as an enzyme-linked immunosorbent assay (ELISA), an immunohistochemical assay, an immunocytochemical assay, and/or a flow cytometry assay of antibody-labeled cells. In certain embodiments, the cancer-associated marker comprises an amino acid sequence set forth in any one of SEQ ID NOs: 3, 6, 9, 13-14 and 17 or a polypeptide sequence encoded by a polynucleotide sequence set forth in any one of SEQ ID NOs: 1-2, 4-5, 7-8, 10-12, 15-16 and 18.

In another aspect, methods are provided for monitoring the progression of a cancer in a patient comprising the steps of: (a) detecting the level of expression in a biological sample from the patient of one or more cancer-associated markers selected from the group consisting of BL1792, BL1794, BL1798, BL1799, BL1801, and BL1811; (b) repeating step (a) using a biological sample from the patient at a subsequent point in time; and, (c) comparing the level of expression detected in step (a) for each marker with the level of expression detected in step (b) for each marker. Using such an approach, a level of expression that is found to be increased at the subsequent point in time may be indicative of the presence of an increased number of cancer cells in the biological sample, which may be indicative of cancer progression in the patient from whom the biological sample was derived. Alternatively, a level of expression that is found to be decreased at the subsequent point in time may be indicative of the presence of fewer cancer cells in the biological sample, which may be indicative of a reduction of disease in the patient from whom the biological sample was derived.

In related aspects, methods are provided for monitoring the treatment of a cancer in a patient comprising the steps of: (a) detecting the level of expression in a biological sample from the patient of one or more cancer-associated markers selected from the group consisting of BL1792, BL1794, BL1798, BL1799, BL1801, and BL1811; (b) repeating step (a) using a biological sample from the patient at a subsequent point in time; and, (c) comparing the level of expression detected in step (a) for each marker with the level of expression detected in step (b) for each marker. Using such an approach, a level of expression that is found to be increased at the subsequent point in time may be indicative of the presence of an increased number of cancer cells in the biological sample, which may be indicative of poor treatment responsiveness of the patient from whom the biological sample was derived. Alternatively, a level of expression that is found to be decreased at the subsequent point in time may be indicative of the presence of fewer cancer cells in the biological sample, which may be indicative of therapeutic responsiveness of the patient from whom the biological sample was derived.

The present invention further provides methods for detecting the presence of cancer cells in a biological sample comprising the steps of: contacting the biological sample with one or more polypeptides selected from the group consisting of the amino acid sequences set forth in SEQ ID NOs: 3, 6, 9, 13-14 and 17 or a polypeptide sequence encoded by a polynucleotide sequence set forth in any one of SEQ ID NOs: 1-2, 4-5, 7-8, 10-12, 15-16 and 18; and, detecting the presence of antibodies in the biological sample that are specific for any one or more of the polypeptides; wherein the presence of antibodies specific for one or more of the polypeptides is indicative of the presence of cancer cells in the biological sample. In this regard, the antibodies are specific for only one polypeptide but multiple antibodies, each specific for one cancer-associated polypeptide, may be detected. Methods for detecting the presence of antibodies specific for a given polypeptide may include any of a variety of well-known and established techniques, illustrative examples of which are described herein.

These and other aspects of the present invention will become apparent upon reference to the following detailed description.

### BRIEF DESCRIPTION OF SEQUENCE IDENTIFIERS

SEQ ID NO:1 is the cDNA sequence for BL1792P BLAM26 B2 FL.

SEQ ID NO:2 is the cDNA sequence for BL1792P ORF.

SEQ ID NO:3 is the amino acid sequence for BL1792P BLAM26 B2 FL.

SEQ ID NO:4 is the cDNA sequence for BL1794 R0 BLAM26 D3 FL.

SEQ ID NO:5 is the cDNA sequence for BL1794P ORF.

SEQ ID NO:6 is the amino acid sequence for BL1794P BLAM26 D3 FL.

SEQ ID NO:7 is the cDNA sequence for BL1798P BLAM27 D7 FL.

SEQ ID NO:8 is the cDNA sequence for BL1798P ORF.

SEQ ID NO:9 is the amino acid sequence for BL1798P BLAM27 D7 FL.

SEQ ID NO:10 is the cDNA sequence for BL1799P BLAM 25 C3 FL. This sequence is the complete cDNA for the human, parathyroid-like protein.

SEQ ID NO:11 is the cDNA sequence for BL1799P ORF1.

SEQ ID NO:12 is the cDNA sequence for BL1799P ORF2.

SEQ ID NO:13 is the amino acid sequence for BL1799P ORF1, encoded by the polynucleotide of SEQ ID NO:11.

SEQ ID NO:14 is the amino acid sequence for BL1799P ORF2, encoded by the polynucleotide of SEQ ID NO:12.

SEQ ID NO:15 is the cDNA sequence for BL1801P BLAM35 C1 FL.

SEQ ID NO:16 is the cDNA sequence for BL1801P ORF.

SEQ ID NO:17 is the amino acid sequence for BL1801P BLAM 35 C1 FL.

SEQ ID NO:18 is the cDNA sequence for BL1811P BLAM31 A10 GS.

### DETAILED DESCRIPTION OF THE INVENTION

The present invention is directed generally to compositions and their use in the diagnosis of cancer, particularly bladder cancer. As described further below, illustrative compositions of the present invention include, but are not restricted to, polynucleotides, oligonucleotide primers and probes, polypeptides and fragments thereof, antibodies and other binding agents. The present invention also provides kits and arrays comprising polynucleotides, oligonucleotide primers and probes, polypeptides and fragments thereof, and antibodies as described herein.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

As used in this specification and the appended claims, the singular forms “a,” “an” and “the” include plural references unless the content clearly dictates otherwise.

Certain terms are defined in the specification. Unless indicated or defined otherwise, all scientific and technical terms used herein have the same meaning as commonly understood by those skilled in the relevant art. General definitions of many terms used herein are provided in: Dictionary of Microbiology and Molecular Biology, 2nd ed. (Singleton et al., 1994, John Wiley & Sons, New York, N.Y.); The Harper Collins Dictionary of Biology (Hale & Marham, 1991, Harper Perennial, New York, N.Y.); and, Dorland's Illustrated Medical Dictionary, 27th ed. (W. A. Dorland, 1988, W. B. Saunders Co., Philadelphia, Pa.).

Cancer-Associated Markers

As noted above, the present invention relates generally to compositions and methods for detecting cancer cells in a biological sample, as well as diagnosing and monitoring cancer in the patient from whom the biological sample was derived, by evaluating the expression of one or more cancer-associated polynucleotide and/or polypeptide sequences. More particularly, the present invention relates to the evaluation in a biological sample of the expression of one or more cancer-associated sequences described herein and referred to as BL1792, BL1794, BL1798, BL1799, BL1801, and BL1811.

By “cancer-associated marker” is meant a polynucleotide or polypeptide sequence of the present invention that is expressed in a substantial proportion of bladder tumor samples, for example greater than about 20%, about 30%, and in certain embodiments, greater than about 50% or more, of bladder tumor samples tested, at a level that is at least two fold, and in certain embodiments, at least five fold, greater than the level of expression in normal tissues, as determined using a representative assay provided herein. A sequence shown to have an increased level of expression in tumor cells has particular utility as a cancer diagnostic marker as further described herein.

It should be noted that in certain embodiments, the cancer-associated sequences of the present invention are tissue-specific sequences as opposed to tumor-specific sequences in that they may be expressed in, for example, normal bladder tissue and bladder tumor tissue. Thus, in general, a cancer-associated sequence should be present at a level that is at least two-fold, preferably three-fold, and more preferably five-fold or higher in tumor tissue than in normal tissue of the same type from which the tumor arose. Expression levels of a particular cancer-associated sequence in tissue types different from that in which the tumor arose are irrelevant in certain diagnostic embodiments since the presence of tumor cells can be confirmed by observation of predetermined differential expression levels, e.g., 2-fold, 5-fold, etc, in tumor tissue to expression levels in normal tissue of the same type. However, other differential expression patterns can be utilized advantageously for diagnostic purposes. For example, in one aspect of the invention, overexpression of a cancer-associated sequence of the invention in tumor tissue and normal tissue of the same type, but not in other normal tissue types, e.g., PBMCs, can be exploited diagnostically. In such a scenario, the presence of metastatic tumor cells, for example in a sample taken from the circulation or from some other tissue site different from that in which the tumor arose, can be identified and/or confirmed by detecting expression of the cancer-associated sequence in the sample, for example using any of a variety of amplification methods as described herein. In this setting, expression of the cancer-associated sequence in normal tissue of the same type in which the tumor arose, does not affect its diagnostic utility.

The present invention, in other aspects, provides isolated cancer-associated polynucleotides. “Isolated,” as used herein, means that a polynucleotide is substantially away from other coding sequences, and that a DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

By “nucleotide sequence”, “nucleic acid sequence” or “polynucleotide” is meant the sequence of nitrogenous bases along a linear information-containing molecule (e.g., DNA or RNA; including cDNA and various forms of RNA such as mRNA, tRNA, hnRNA, and the like) that is capable of hydrogen-bonding with another linear information-containing molecule having a complementary base sequence. The terms are not meant to limit such information-containing molecules to polymers of nucleotides per se but are also meant to include molecular structures containing one or more nucleotide analogs or abasic subunits in the polymer. The polymers may include base subunits containing a sugar moiety or a substitute for the ribose or deoxyribose sugar moiety (e.g., 2′ halide- or methoxy-substituted pentose sugars), and may be linked by linkages other than phosphodiester bonds (e.g., phosphorothioate, methylphosphonate or peptide linkages).

As will be understood by those skilled in the art, the cancer-associated polynucleotides of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include hnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein of the invention or a portion thereof) or may comprise a sequence that encodes a variant or derivative, of such a sequence.

Therefore, according to another aspect of the present invention, polynucleotide compositions are provided that comprise some or all of a polynucleotide sequence set forth in any one of SEQ ID NOs: 1-2, 4-5, 7-8, 10-12, 15-16 and 18, the complement of a polynucleotide sequence set forth in any one of SEQ ID NOs: 1-2, 4-5, 7-8, 10-12, 15-16 and 18, and degenerate variants of a polynucleotide sequence set forth in any one of SEQ ID NOs: 1-2, 4-5, 7-8, 10-12, 15-16 and 18. In a related embodiment, the invention provides polynucleotides encoding any one of the cancer-associated polypeptides recited in SEQ ID NOs: 3, 6, 9, 13-14 and 17, or the complement thereof.

In other related embodiments, the present invention provides polynucleotide variants having substantial identity to the sequences disclosed herein in SEQ ID NOs: 1-2, 4-5, 7-8, 10-12, 15-16 and 18, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In additional embodiments, the present invention provides polynucleotide fragments comprising or consisting of various lengths of contiguous stretches of sequence identical to or complementary to one or more of the cancer-associated polynucleotides disclosed herein. For example, polynucleotides are provided by this invention that comprise or consist of at least about 10, 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence.

The present invention further provides oligonucleotides and compositions comprising oligonucleotides. By “oligonucleotide” is meant a polymeric chain of two or more chemical subunits, each subunit comprising a nucleotide base moiety, a sugar moiety, and a linking moiety that joins the subunits in a linear spacial configuration. An oligonucleotide may contain up to thousands of such subunits, but generally contains subunits in a range having a lower limit of between about 5 to about 10 subunits, and an upper limit of between about 20 to about 1,000 subunits. The most common nucleotide base moieties are guanine (G), adenine (A), cytosine (C), thymine (T) and uracil (U), although other rare or modified nucleotide bases able to form hydrogen bonds (e.g., inosine (I)) are well known to those skilled in the art. The most common sugar moieties are ribose and deoxyribose, although 2′-O-methyl ribose, halogenated sugars, and other modified and different sugars are well known. The linking group is usually a phosphorus-containing moiety, commonly a phosphodiester linkage, although other known phosphate-containing linkages (e.g., phosphorothioates or methylphosphonates) and non-phosphorus-containing linkages (e.g., peptide-like linkages found in “peptide nucleic acids” or PNAs) known in the art are included. Likewise, an oligonucleotide includes one in which at least one base moiety has been modified, for example, by the addition of propyne groups, so long as: (1) the modified base moiety retains the ability to form a non-covalent association with G, A, C, T or U; and, (2) an oligonucleotide comprising at least one modified nucleotide base moiety is not sterically prevented from hybridizing with a complementary single-stranded nucleic acid. An oligonucleotide's ability to hybridize with a complementary nucleic acid strand under particular conditions (e.g., temperature or salt concentration) is governed by the sequence of base moieties, as is well-known to those skilled in the art (Sambrook, J. et al., 1989, Molecular Cloning, A Laboratory Manual, 2nd ed. (Cold Spring Harbor Laboratory Press, Cold Spring Harbor, N.Y.), particularly pp. 7.37-7.57 and 11.47-11.57). Thus, oligonucleotides can comprise 10, 15, 20, 25, 30, 35, 40, 45, 50, 55, 60, 65, 70, 75, 80, 85, 90, 95 or 100 subunits. In certain embodiments, the oligonucleotides of the present invention consist of or comprise 10, 15, 20, 25, 30, 35, 40, 45, 50, 55, 60, 65, 70, 75, 80, 85, 90, 95 or 100 contiguous nucleotides of any one of the polynucleotides recited in SEQ ID NOs: 1-2, 4-5, 7-8, 10-12, 15-16 and 18. In further embodiments, the oligonucleotides of the present invention comprise no more than 10, 15, 20, 25, 30, 35, 40, 45, 50, 55, 60, 65, 70, 75, 80, 85, 90, 95 or 100 contiguous nucleotides of any one of the polynucleotides recited in SEQ ID NOs: 1-2, 4-5, 7-8, 10-12, 15-16 and 18 and may also comprise additional nucleotides unrelated to the polynucleotides recited in SEQ ID NOs: 1-2, 4-5, 7-8, 10-12, 15-16 and 18. For example, as would be readily recognized by the skilled artisan, oligonucleotide primers and probes can also comprise additional sequence unrelated to the target nucleic acid, such as restriction endonuclease cleavage sites, linkers, and the like. This additional sequence may consist of 1, 2, 3, 4, 5, 6, 7, 8, 9, 10, 11, 12, 13, 14, 15, 16, 17, 18, 19, or 20, or more nucleotides at either end of the disclosed sequence or at both ends of the disclosed sequence.

The present invention also provides cancer-associated polypeptides. As used herein, the term “polypeptide” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. In certain embodiments, polypeptides of interest in the context of this invention are amino acid subsequences comprising epitopes, e.g., antigenic determinants recognized by antibodies.

Particularly illustrative polypeptides of the present invention comprise those encoded by a polynucleotide sequence set forth in any one of SEQ ID NOs: 1-2, 4-5, 7-8, 10-12, 15-16 and 18. Certain other illustrative polypeptides of the invention comprise amino acid sequences as set forth in any one of SEQ ID NOs: 3, 6, 9, 13-14 and 17.

The polypeptides of the present invention are sometimes herein referred to as “bladder cancer-associated proteins”, “bladder cancer-associated markers”, or “bladder tumor polypeptides”, as an indication that their identification has been based at least in part upon their increased levels of expression in bladder tumor samples. Thus, a “bladder cancer-associated polypeptide” or “bladder tumor protein,” refers generally to a polypeptide sequence of the present invention that is expressed in a substantial proportion of bladder tumor samples, for example preferably greater than about 20%, more preferably greater than about 30%, and most preferably greater than about 50% or more of bladder tumor samples tested, at a level that is at least two fold, and preferably at least five fold, greater than the level of expression in normal tissues, as determined using a representative assay provided herein. A bladder cancer-associated polypeptide sequence of the invention, based upon its increased level of expression in tumor cells, has particular utility both as a diagnostic marker as well as a therapeutic target, as further described below.

In certain embodiments, the polypeptides of the invention are immunogenic in that they react detectably within an immunoassay (such as an ELISA) with antisera from a patient with bladder cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” or polypeptide “fragment” as used herein, is a fragment of a polypeptide of the invention that itself is immunologically reactive (i.e., specifically binds) with antibodies that recognize the full-length polypeptide. Such polypeptide fragments may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies or antisera. Further techniques include epitope mapping using overlapping peptides and peptide pools that encompass an entire cancer-associated polypeptide sequence. As used herein, antisera and antibodies are “antigen-specific” if they specifically bind to an antigen (i.e., they react with the protein in an ELISA or other immunoassay, and do not react in a statistically significant manner under similar conditions with suitable control proteins). Such antisera and antibodies may be prepared as described herein, and using well-known techniques.

In one embodiment, an immunogenic portion of a polypeptide of the present invention is a fragment that reacts with antisera and/or monoclonal antibodies at a level that is not statistically significantly less than the reactivity of the full-length polypeptide (e.g., in an ELISA or similar immunoassay). In this manner, fragments of a cancer-associated polypeptide as disclosed herein can be used in lieu of a full-length polypeptide in any number of methods for detecting bladder cancer as described herein. Preferably, the level of immunogenic activity of the immunogenic portion is at least about 50%, preferably at least about 70% and most preferably greater than about 90% of the immunogenicity for the full-length polypeptide. In some instances, polypeptide fragments useful in the present invention will be identified that have a level of reactivity greater than that of the corresponding full-length polypeptide, e.g., having greater than about 100% or 150% or more immunogenic activity. Thus, the present invention provides polypeptide fragments comprising at least about 5, 10, 15, 20, 25, 30, 35, 40, 45, 50, 55, 60, 65, 70, 75, 80, 85, 90, 95 or 100 contiguous amino acids, or more, including all intermediate lengths, of a cancer-associated polypeptide set forth herein, such as those set forth in SEQ ID NOs: 3, 6, 9, 13-14 and 17, or those encoded by a polynucleotide sequence set forth in a sequence of SEQ ID NOs: 1-2, 4-5, 7-8, 10-12, 15-16 and 18. In certain embodiments, the present invention provides polypeptide fragments that consist of no more than about 5, 10, 15, 20, 25, 30, 35, 40, 45, 50, 55, 60, 65, 70, 75, 80, 85, 90, 95 or 100 contiguous amino acids, including all intermediate lengths, of a cancer-associated polypeptide set forth herein, such as those set forth in SEQ ID NOs: 3, 6, 9, 13-14 and 17, or those encoded by a polynucleotide sequence set forth in a sequence of SEQ ID NOs: 1-2, 4-5, 7-8, 10-12, 15-16 and 18 and may also comprise additional amino acids unrelated to the polypeptides recited in SEQ ID NOs: 3, 6, 9, 13-14 and 17. For example, as would be readily recognized by the skilled artisan, polypeptide fragments such as antibody epitopes can also comprise additional sequence for use in purification or attachment to solid surfaces as described herein (e.g., His tags or other similar tags). This additional sequence may consist of 1, 2, 3, 4, 5, 6, 7, 8, 9, 10, 11, 12, 13, 14, 15, 16, 17, 18, 19, or 20, or more amino acids at either end of the fragment of interest or at both ends of the fragment of interest.

In another embodiment of the invention, recombinant polypeptides are provided that comprise one or more fragments that are specifically recognized by antibodies that are immunologically reactive with one or more cancer-associated polypeptides described herein.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein. The polypeptide variants provided by the present invention are immunologically reactive with an antibody that reacts with the corresponding non-variant full-length cancer-associated polypeptide as set forth in SEQ ID NOs: 3, 6, 9, 13-14 and 17. In certain embodiments, the polypeptide variants provided by the present invention exhibit a level of immunogenic activity of at least about 50%, preferably at least about 70%, and most preferably at least about 90% or more of that exhibited by a non-variant polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., which is specifically bound by antibodies that specifically bind the parent polypeptide. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their utility in, for example, detection of bladder cancer.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Praline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

When comparing polypeptide or polynucleotide sequences, two sequences are said to be “identical” if the nucleotide or amino acid sequence in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O., (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes, pp. 626-645 Methods in Enzymology vol. 183, Academic Press, Inc., San Diego, Calif.; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Saitou, N. Nei, M., Mol. Biol. Evol. 4:406-425 (1987); Sneath, P.H.A. and Sokal, R. R., Numerical Taxonomy—the Principles and Practice of Numerical Taxonomy, Freeman Press, San Francisco, Calif. (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide or polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid or nucleic acid residue occurs in both sequences to yield the number of matched positions, dividing the number of matched positions by the total number of positions in the reference sequence (i.e., the window size) and multiplying the results by 100 to yield the percentage of sequence identity.

Binding Agents

The present invention also provides for binding agents that specifically bind to the cancer-associated polynucleotides and polypeptides disclosed herein. Such binding agents may be used in the methods of the invention for detecting the presence and/or level of BL1792, BL1794, BL1798, BL1799, BL1801, and BL1811 polypeptide and polynucleotide expression in biological samples (including tissue sections) using representative assays either illustratively described herein or known and available in the art.

A binding agent used according to this aspect of the invention can include essentially any binding agent having sufficient specificity and affinity for the cancer-associated markers described herein to facilitate the detection and identification of the markers in a biological sample. For example, by way of illustration, a binding agent may be an antibody, an antigen-binding fragment of an antibody, a ribosome, with or without a peptide component, an RNA molecule, or a polypeptide. In one illustrative example, a binding agent is an agent identified via phage display library screening to specifically bind a cancer-associated marker described herein.

Certain preferred binding agents for use according to the present invention include antibodies or antigen-binding fragments thereof that specifically bind a cancer-associated marker described herein. An antibody or antigen-binding fragment thereof is said to “specifically bind” to a polypeptide of the invention if it reacts at a detectable level (within, for example, an ELISA) with the polypeptide but does not react with a biologically unrelated polypeptide in any statistically significant fashion under the same or similar conditions. Specific binding, as used in this context, generally refers to the non-covalent interactions of the type that occur between an immunoglobulin molecule and an antigen for which the immunoglobulin is specific. The strength or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and the geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity and is thus equal to the dissociation constant K_{d}. See, generally, Davies et al. (1990) Annual Rev. Biochem. 59:439-473.

An “antigen-binding site” or “binding portion” of an antibody refers to the part of the immunoglobulin molecule that participates in antigen binding. The antigen-binding site is formed by amino acid residues of the N-terminal variable (V) regions of the heavy (H) and light (L) chains. Three highly divergent stretches within the variable regions of the heavy and light chains are referred to as “hypervariable regions.” These hypervariable regions are interposed between more conserved flanking stretches known as “framework regions” (FRs). Thus, the term “FR” refers to amino acid sequences naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen. The three hypervariable regions of each of the heavy and light chains are referred to as “complementarity-determining regions” (CDRs).

In one embodiment, antibodies or other binding agents that bind to a cancer-associated marker described herein will preferably generate a signal indicating the presence of a cancer in at least about 20%, 30% or 50% of samples and/or patients with the disease. Biological samples (e.g., blood, sera, sputum, urine and/or tumor biopsies) from patients with and without a cancer (as determined using standard clinical tests) may be assayed as described herein for the presence of polypeptides that bind to the binding agent.

In one preferred embodiment, a binding agent is an antibody or an antigen-binding fragment thereof. Antibodies may be prepared by any of a variety of techniques known to those of ordinary skill in the art (see, e.g., Harlow and Lane, Antibodies: A Laboratory Manual, Cold Spring Harbor Laboratory, 1988; Ausubel et al., Current Protocols in Molecular Biology, John Wiley & Sons, New York (2001 and later updates thereto)). Illustrative methods for the production of antibodies generally involve the use of a polypeptide, produced by either recombinant or synthetic approaches, as an immunogen. In order to produce a desired recombinant polypeptide, a nucleotide sequence encoding the polypeptide, or functional equivalents, may be inserted into an appropriate expression vector, i.e., a vector which contains the necessary elements for the transcription and translation of the inserted coding sequence. Methods which are well-known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in: Sambrook, J. et al. (1989) Molecular Cloning, A Laboratory Manual, Cold Spring Harbor Press, Plainview, N.Y.; and, Ausubel, F. M. et al. Eds. (2001 and later updates thereto) Current Protocols in Molecular Biology, John Wiley & Sons, New York, N.Y.

A variety of expression vector/host systems may be utilized to contain and express polynucleotide sequences. These include, but are not limited to: microorganisms, such as bacteria, transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or bacterial expression vectors (e.g., Ti or pBR322 plasmids); and, animal cell systems. These and other suitable expression systems for the production of recombinant polypeptides are known in the art and may be used in the practice of the present invention.

In addition to recombinant production methods, peptide and/or polypeptides may be synthesized, in whole or in part, using chemical methods well-known in the art (see Caruthers, M. H. et al. (1980) Nucl. Acids Res. Symp. Ser. 215-223, Horn, T. et al. (1980) Nucl. Acids Res. Symp. Ser. 225-232). For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.). A newly synthesized peptide may be substantially purified by preparative HPLC (e.g., Creighton, T. (1983) Proteins, Structures and Molecular Principles, WH Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In certain embodiments, antibodies can be produced by cell culture techniques, including the generation of monoclonal antibodies as described herein, or via transfection of antibody genes into suitable bacterial or mammalian cell hosts in order to allow for the production of recombinant antibodies. In one technique, an immunogen comprising a polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for a polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized, for example, by fusion with a myeloma cell fusion partner, preferably one that is syngeneic with the immunized animal. A variety of fusion techniques may be employed. For example, the spleen cells and myeloma cells may be combined with a non-ionic detergent for a few minutes and then plated at low density on a selective medium that supports the growth of hybrid cells but not myeloma cells. One illustrative selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J. Immunol. 138:4534-4538; and, Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and, Jones et al. (1986) Nature 321:522-525), and rodent CDRs supported by recombinantly veneered rodent FRs (European Patent No. 0 519 596). These “humanized” molecules are designed to minimize unwanted immunological response toward rodent anti-human antibody molecules.

Kits and Arrays for the Detection of Bladder Cancer-Associated Markers

The present invention also provides diagnostic kits comprising oligonucleotides, polypeptides, or binding agents such as antibodies, as described herein. Components of such diagnostic kits may be compounds, reagents, detection reagents, reporter groups, containers and/or equipment.

The kits described herein may include detection reagents and reporter groups. Reporter groups may include radioactive groups, dyes, fluorophores, biotin, calorimetric substrates, enzymes, or colloidal compounds. Illustrative reporter groups include but are not limited to, fluorescein, tetramethyl rhodamine, Texas Red, coumarins, carbonic anhydrase, urease, horseradish peroxidase, dehydrogenases and/or colloidal gold or silver. For radioactive groups, scintillation counting or autoradiographic methods are generally appropriate for detection. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

In one embodiment, a kit may be designed to detect the level of mRNA encoding a cancer-associated protein in a biological sample. Such kits generally comprise at least one oligonucleotide probe or primer, as described herein, that specifically hybridizes to a cancer-associated polynucleotide. Such an oligonucleotide may be used, for example, within an amplification or hybridization assay. Additional components that may be present within such kits include restriction enzymes, reverse transcriptases, polymerases, ligases, linkers, nucleoside triphosphates, suitable buffers, labels, and/or other accessories, a second or multiple oligonucleotides and/or detection reagents or container to facilitate the detection of a cancer-associated nucleic acid.

Kits of the invention may include one or more oligonucleotide primers or probes specific for a cancer-associated polynucleotide of interest such as the polynucleotides comprising the nucleic acid sequences as set forth in SEQ ID NOs: 1-2, 4-5, 7-8, 10-12, 15-16 and 18 or a polynucleotide encoding any one of the cancer-associated polypeptides recited in SEQ ID NOs: 3, 6, 9, 13-14 and 17, or the complement thereof. In certain embodiments, the kits of the invention the diagnostic kits for detecting bladder cancer cells in a biological sample comprising at least two oligonucleotide primers specific for any one of the cancer-associated polynucleotides recited in SEQ ID NOs: 1-2, 4-5, 7-8, 10-12, 15-16 and 18 a polynucleotide encoding any one of the cancer-associated polypeptides recited in SEQ ID NOs: 3, 6, 9, 13-14 and 17, or the complement thereof. In certain embodiments, the kits of the invention comprise at least two, three, four, five, six, or more, oligonucleotide primer pairs, for example for use with an amplification method as described herein, each pair being specific for one of the cancer-associated polynucleotides described herein.

Kits may also comprise one or more positive controls, one or more negative controls, and a protocol for identification of the cancer-associated sequence of interest using any one of the amplification or hybridization assays as described herein. In certain embodiments, one or more oligonucleotide primers or probes are immobilized on a solid support. A negative control may include a nucleic acid (e.g., cDNA) molecule encoding a sequence other than the cancer-associated sequence of interest. The negative control nucleic acid may be a naked nucleic acid (e.g., cDNA) molecule or inserted into a bacterial cell. In certain embodiments, the negative control nucleic acid is double stranded, however, a single stranded nucleic acid may be employed. In certain embodiments, the negative control comprises a suitable buffer containing no nucleic acid. A positive control may include the nucleic acid (e.g., cDNA) sequence of the cancer-associated sequence of interest, or a portion thereof. The positive control nucleic acid may be a naked nucleic acid molecule or inserted into a bacterial cell, for example. In certain embodiments, the positive control nucleic acid is double stranded, however, a single stranded nucleic acid may be employed. Typically, the nucleic acid is obtained from a bacterial lysate using techniques known in the art. In certain embodiments, the positive control comprises a set of oliognucleotide primers or a probe suitable for amplifying or otherwise hybridizing to an internal control always present in the biological sample to be tested, such as primers or probes specific for any of a variety of housekeeping genes.

In a further embodiment, the kits of the present invention comprise one or more cancer-associated polypeptides or a fragment thereof wherein the fragment is specifically bound by antibodies that are specific for the full-length cancer-associated polypeptide. The kits may contain at least two, three, four, five, or more cancer-associated polypeptides or fragments thereof. In this regard, the cancer-associated polypeptides, or fragments thereof, may be provided attached to a support material, as described herein or in an appropriate buffer. One or more additional containers may enclose elements, such as reagents or buffers, to be used in any of a variety of detection assays as described herein. Such kits may also, or alternatively, contain a detection reagent that contains a reporter group suitable for direct or indirect detection of antibody binding.

In a further embodiment, the kits of the invention comprise one or more monoclonal antibodies or antigen-binding fragments thereof that specifically bind to a cancer-associated protein as described herein. In certain embodiments, a kit may comprise at least two, three, four, five, six, or seven monoclonal antibodies or antigen-binding fragments thereof, each specific for any one of the cancer-associated polypeptides disclosed herein. Such antibodies or antigen-binding fragments thereof may be provided attached to a support material, as described herein. One or more additional containers may enclose elements, such as reagents or buffers, to be used in any of a variety of detection assays as described herein. Such kits may also, or alternatively, contain a detection reagent as described above that contains a reporter group suitable for direct or indirect detection of antibody binding or a detection reagent suitable for detection of nucleic acid.

In certain embodiments, the binding agents as described herein, such as antibodies, polypeptides, or polynucleotides, are arranged on an array.

In one embodiment, the panel is an addressable array. As such, the addressable array may comprise a plurality of distinct binding agents, such as antibodies, polypeptides, or polynucleotides, attached to precise locations on a solid phase surface, such as a plastic chip. The position of each distinct binding agent on the surface is known and therefore “addressable”. In one embodiment, the binding agents are distinct antibodies that each has specific affinity for one of the cancer-associated polypeptides set forth herein.

In one embodiment, the binding agents, such as antibodies, are covalently linked to the solid surface, such as a plastic chip, for example, through the Fc domains of antibodies. In another embodiment, antibodies are adsorbed onto the solid surface. In a further embodiment, the binding agent, such as an antibody, is chemically conjugated to the solid surface. In a further embodiment, the binding agents are attached to the solid surface via a linker. In certain embodiments, detection with multiple specific binding agents is carried out in solution.

Methods of constructing protein arrays, including antibody arrays, are known in the art (see, e.g., U.S. Pat. No. 5,489,678; U.S. Pat. No. 5,252,743; Blawas and Reichert, 1998, Biomaterials 19:595-609; Firestone et al., 1996, J. Amer. Chem. Soc. 18, 9033-9041; Mooney et al., 1996, Proc. Natl. Acad. Sci. 93, 12287-12291; Pirrung et al, 1996, Bioconjugate Chem. 7, 317-321; Gao et al, 1995, Biosensors Bioelectron 10, 317-328; Schena et al, 1995, Science 270, 467-470; Lom et al., 1993, J. Neurosci. Methods, 385-397; Pope et al., 1993, Bioconjugate Chem. 4, 116-171; Schramm et al., 1992, Anal. Biochem. 205, 47-56; Gombotz et al., 1991, J. Biomed. Mater. Res. 25, 1547-1562; Alarie et al., 1990, Analy. Chim. Acta 229, 169-176; Owaku et al, 1993, Sensors Actuators B, 13-14, 723-724; Bhatia et al., 1989, Analy. Biochem. 178, 408-413; Lin et al., 1988, IEEE Trans. Biomed. Engng., 35(6), 466-471).

In one embodiment, the binding agents, such as antibodies, are arrayed on a chip comprised of electronically activated copolymers of a conductive polymer and the detection reagent. Such arrays are known in the art (see e.g., U.S. Pat. No. 5,837,859 issued Nov. 17, 1998; PCT publication WO 94/22889 dated Oct. 13, 1994). The arrayed pattern may be computer generated and stored. The chips may be prepared in advance and stored appropriately. The antibody array chips can be regenerated and used repeatedly.

Methods of constructing polynucleotide arrays are known in the art. Techniques for constructing arrays and methods of using these arrays are described, for example, in U.S. Pat. Nos. 5,593,839, 5,578,832, 5,599,695, 5,556,752, and 5,631,734.

Methods for Detecting Bladder Cancer-Associated Markers

The present invention provides for a variety of methods for the detection of the cancer-associated markers disclosed herein. The cancer-associated sequences of the invention may be used in the detection of essentially any cancer type that expresses one or more such sequences. In one particular embodiment of the invention, the cancer-associated sequences described herein have been found particularly advantageous in the detection of bladder cancer.

According to one aspect of the invention, methods are provided for detecting the presence of cancer cells in a biological sample comprising the steps of: detecting the level of expression in the biological sample of at least one cancer-associated marker, wherein the cancer-associated marker comprises a polynucleotide set forth in any one of SEQ ID NOs: 1-2, 4-5, 7-8, 10-12, 15-16 and 18 or a polypeptide set forth in any one of SEQ ID NOs: 3, 6, 9, 13-14 and 17; and, comparing the level of expression detected in the biological sample for the cancer-associated marker to a predetermined cut-off value for the cancer-associated marker; wherein a detected level of expression above the predetermined cut-off value for the cancer-associated marker is indicative of the presence of cancer cells in the biological sample.

In certain embodiments, the methods of the invention detect the expression of any one or more of BL1792, BL1794, BL1798, BL1799, BL1801, and BL1811 mRNA in biological samples. Expression of the cancer-associated sequences of the invention may be detected at the mRNA level using methodologies well-known and established in the art, including, for example, in situ and in vitro hybridization, and/or any of a variety of nucleic acid amplification methods, as further described herein.

Alternatively, or additionally, the methods described herein can detect the expression of BL1792, BL1794, BL1798, BL1799, BL1801, and BL1811 polypeptides in a biological sample using methodologies well-known and established in the art, including, for example, ELISA, immunohistochemistry, immunocytochemistry, flow cytometry and/or other known immunoassays, as further described herein.

Essentially any biological sample suspected of containing cancer-associated markers, antibodies to such cancer-associated markers and/or cancer cells expressing such markers or antibodies may be used for the methods of the invention. For example, the biological sample can be a tissue sample, such as a tissue biopsy sample, known or suspected of containing cancer cells. The biological sample may be derived from a tissue suspected of being the site of origin of a primary tumor. Alternatively, the biological sample may be derived from a tissue or other biological sample distinct from the suspected site of origin of a primary tumor in order to detect the presence of metastatic cancer cells in the tissue or sample that have escaped the site of origin of the primary tumor. In certain embodiments, the biological sample is a tissue biopsy sample derived from tissue of the bladder. In other embodiments, the biological sample tested according to such methods is selected from the group consisting of a biopsy sample, lavage sample, sputum sample, serum sample, peripheral blood sample, lymph node sample, bone marrow sample, urine sample, and pleural effusion sample.

A predetermined cut-off value used in the methods described herein for determining the presence of cancer can be readily identified using well-known techniques. For example, in one illustrative embodiment, the predetermined cut-off value for the detection of cancer is the average mean signal obtained when the relevant method of the invention is performed on suitable negative control samples, e.g., samples from patients without cancer. In another illustrative embodiment, a sample generating a signal that is at least two or three standard deviations above the predetermined cut-off value is considered positive.

In another embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In certain embodiments, multiple cancer-associated sequences described herein can be used in combination in a “complementary” fashion to detect bladder cancer. Thus, in certain embodiments, any combination of one or more of BL1792, BL1794, BL1798, BL1799, BL1801, and BL1811 can be used in any of a variety of diagnostic assays as described herein to detect bladder cancer. Thus, in one embodiment 2, 3, 4, 5, or all of the cancer-associated markers described herein can be detected simultaneously to detect bladder cancer.

In this regard, in certain embodiments, the cancer-associated markers described herein can be detected in combination with any known cancer markers in a complementary fashion to detect bladder cancer. In certain embodiments, use of multiple markers may increase the sensitivity and/or specificity of cancers detected.

By “amplification” or “nucleic acid amplification” is meant production of multiple copies of a target nucleic acid that contains at least a portion of the intended specific target nucleic acid sequence (e.g., BL1792, BL1794, BL1798, BL1799, BL1801, and BL1811). The multiple copies may be referred to as amplicons or amplification products. In certain embodiments, the amplified target contains less than the complete target gene sequence (introns and exons) or an expressed target gene sequence (spliced transcript of exons and flanking untranslated sequences). For example, specific amplicons may be produced by amplifying a portion of the target polynucleotide by using amplification primers that hybridize to, and initiate polymerization from, internal positions of the target polynucleotide. In certain embodiments, the amplified portion contains a detectable target sequence that may be detected using any of a variety of well-known methods. In certain embodiments, detection takes place during amplification of a target sequence.

The present invention also provides oligonucleotide primers. By “primer” or “amplification primer” is meant an oligonucleotide capable of binding to a region of a target nucleic acid or its complement and promoting, either directly or indirectly, nucleic acid amplification of the target nucleic acid. In most cases, a primer will have a free 3′ end that can be extended by a nucleic acid polymerase. In certain embodiments, the 3′ end of a promoter-primer, or a subpopulation of such promoter-primers, may be modified to block or reduce primer extension. All amplification primers include a base sequence capable of hybridizing via complementary base interactions to at least one strand of the target nucleic acid or a strand that is complementary to the target sequence. For example, in PCR, amplification primers anneal to opposite strands of a double-stranded target DNA that has been denatured. The primers are extended by a thermostable DNA polymerase to produce double-stranded DNA products, which are then denatured with heat, cooled and annealed to amplification primers. Multiple cycles of the foregoing steps (e.g., about 20 to about 50 thermic cycles) exponentially amplifies the double-stranded target DNA.

A “target-binding sequence” of an amplification primer is the portion that determines target specificity because that portion is capable of annealing to the target nucleic acid strand or its complementary strand but does not detectably anneal to non-target nucleic acid strands under the same conditions. The complementary target sequence to which the target-binding sequence hybridizes is referred to as a primer-binding sequence. For primers or amplification methods that do not require additional functional sequences in the primer (e.g., PCR amplification), the primer sequence consists essentially of a target-binding sequence, whereas other methods (e.g., TMA or SDA) include additional specialized sequences adjacent to the target-binding sequence (e.g., an RNA polymerase promoter sequence adjacent to a target-binding sequence in a promoter-primer or a restriction endonuclease recognition sequence for an SDA primer). It will be appreciated by those skilled in the art that all of the primer and probe sequences of the present invention may be synthesized using standard in vitro synthetic methods. Also, it will be appreciated that those skilled in the art could modify primer sequences disclosed herein using routine methods to add additional specialized sequences (e.g., promoter or restriction endonuclease recognition sequences, linker sequences, and the like) to make primers suitable for use in a variety of amplification methods. Similarly, promoter-primer sequences described herein can be modified by removing the promoter sequences to produce amplification primers that are essentially target-binding sequences suitable for amplification procedures that do not use these additional functional sequences.

By “target sequence” is meant the nucleotide base sequence of a nucleic acid strand, at least a portion of which is capable of being detected using primers and/or probes in the methods as described herein, such as a labeled oligonucleotide probe. Primers and probes bind to a portion of a target sequence, which includes either complementary strand when the target sequence is a double-stranded nucleic acid.

By “equivalent RNA” is meant a ribonucleic acid (RNA) having the same nucleotide base sequence as a deoxyribonucleic acid (DNA) with the appropriate U for T substitution(s). Similarly, an “equivalent DNA” is a DNA having the same nucleotide base sequence as an RNA with the appropriate T for U substitution(s). It will be appreciated by those skilled in the art that the terms “nucleic acid” and “oligonucleotide” refer to molecular structures having either a DNA or RNA base sequence or a synthetic combination of DNA and RNA base sequences, including analogs thereof, which include “abasic” residues.

The term “specific for” in the context of oligonucleotide primers and probes, is a term of art well understood by the skilled artisan to refer to a particular primer or probe capable of annealing/hybridizing/binding to a target nucleic acid or its complement but which primer or probe does not anneal/hybridize/bind to non-target nucleic acid sequences under the same conditions in a statistically significant or detectable manner. Thus, for example, in the setting of an amplification technique, a primer, primer set (e.g., primer pair), or probe that is specific for a target nucleic acid of interest would amplify the target nucleic acid of interest but would not detectably amplify sequences that are not of interest. Note that a primer pair generally for the purposes of amplification comprises a first primer and a second primer wherein the first and second primers specifically hybridize to opposite strands (e.g., sense/antisense, polynucleotide/complement thereof) of a target polynucleotide. As would be recognized by the skilled artisan, primers and probes that are specific for a particular target nucleic acid sequence of interest can be designed using any of a variety of computer programs available in the art (see, e.g., Methods Mol. Biol. 2002; 192:19-29) or can be designed by eye by comparing the nucleic acid sequence of interest to other relevant known sequences. In certain embodiments, the conditions under which a primer or probe is specific for a target nucleic acid of interest can be routinely optimized by changing parameters of the reaction conditions. For example, in PCR, a variety of parameters can be changed, such as annealing or extension temperature, concentration of primer and/or probe, magnesium concentration, the use of “hot start” conditions such as wax beads or specifically modified polymerase enzymes, addition of formamide, DMSO or other similar compounds. In other hybridization methods, conditions can similarly be routinely optimized by the skilled artisan using techniques known in the art.

Many well-known methods of nucleic acid amplification require thermocycling to alternately denature double-stranded nucleic acids and hybridize primers; however, other well-known methods of nucleic acid amplification are isothermal. The polymerase chain reaction (U.S. Pat. Nos. 4,683,195; 4,683,202; 4,800,159; 4,965,188), commonly referred to as PCR, uses multiple cycles of denaturation, annealing of primer pairs to opposite strands, and primer extension to exponentially increase copy numbers of the target sequence. In a variation called RT-PCR, reverse transcriptase (RT) is used to make a complementary DNA (cDNA) from mRNA, and the cDNA is then amplified by PCR to produce multiple copies of DNA. The ligase chain reaction (Weiss, R. 1991, Science 254: 1292), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product. Another method is strand displacement amplification (Walker, G. et al., 1992, Proc. Natl. Acad. Sci. USA 89:392-396; U.S. Pat. Nos. 5,270,184 and 5,455,166), commonly referred to as SDA, which uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (European Pat. No. 0 684 315). Other amplification methods include: nucleic acid sequence based amplification (U.S. Pat. No. 5,130,238), commonly referred to as NASBA; one that uses an RNA replicase to amplify the probe molecule itself (Lizardi, P. et al., 1988, BioTechnol. 6: 1197-1202), commonly referred to as Qβ replicase; a transcription based amplification method (Kwoh, D. et al., 1989, Proc. Natl. Acad. Sci. USA 86:1173-1177); self-sustained sequence replication (Guatelli, J. et al., 1990, Proc. Natl. Acad. Sci. USA 87: 1874-1878); and, transcription mediated amplification (U.S. Pat. Nos. 5,480,784 and 5,399,491 and US Publication No. 2006/46265), commonly referred to as TMA. For further discussion of known amplification methods see Persing, David H., 1993, “In Vitro Nucleic Acid Amplification Techniques” in Diagnostic Medical Microbiology: Principles and Applications (Persing et al., Eds.), pp. 51-87 (American Society for Microbiology, Washington, D.C.).

Illustrative transcription-based amplification systems of the present invention include TMA, which employs an RNA polymerase to produce multiple RNA transcripts of a target region (U.S. Pat. Nos. 5,480,784 and 5,399,491; and US Publication No. 2006/46265). TMA uses a “promoter-primer” that hybridizes to a target nucleic acid in the presence of a reverse transcriptase and an RNA polymerase to form a double-stranded promoter from which the RNA polymerase produces RNA transcripts. These transcripts can become templates for further rounds of TMA in the presence of a second primer capable of hybridizing to the RNA transcripts. Unlike PCR, LCR or other methods that require heat denaturation, TMA is an isothermal method that uses an RNase H activity to digest the RNA strand of an RNA:DNA hybrid, thereby making the DNA strand available for hybridization with a primer or promoter-primer. Generally, the RNase H activity associated with the reverse transcriptase provided for amplification is used.

By “nucleic acid amplification conditions” is meant environmental conditions, including salt concentration, temperature, the presence or absence of temperature cycling, the presence of a nucleic acid polymerase, nucleoside triphosphates, and cofactors, that are sufficient to permit the production of multiple copies of a target nucleic acid or its complementary strand using a nucleic acid amplification method.

By “detecting” an amplification product is meant any of a variety of methods for determining the presence of an amplified nucleic acid, such as, for example, hybridizing a labeled probe to a portion of the amplified product. A labeled probe is an oligonucleotide that specifically binds to another sequence and contains a detectable group that may be, for example, a fluorescent moiety, chemiluminescent moiety, radioisotope, biotin, avidin, enzyme, enzyme substrate, or other reactive group. In certain embodiments, a labeled probe includes an acridinium ester (AE) moiety that can be detected chemiluminescently under appropriate conditions (as described, e.g., in U.S. Pat. No. 5,283,174). Other well-known detection techniques include, for example, gel filtration, gel electrophoresis and visualization of the amplicons, and High Performance Liquid Chromatography (HPLC). In certain embodiments, for example using real-time TMA or real-time PCR, the level of amplified product is detected as the product accumulates. The detecting step may either be qualitative or quantitative, although quantitative detection of amplicons may be preferred, as the level of gene expression may be indicative of the degree of metastasis, recurrence of cancer and/or responsiveness to therapy.

Assays for purifying and detecting a target cancer-associated polynucleotide often involve capturing a target polynucleotide on a solid support. The solid support retains the target polynucleotide during one or more washing steps of a target polynucleotide purification procedure. One technique involves capture of the target polynucleotide by a polynucleotide fixed to a solid support and hybridization of a detection probe to the captured target polynucleotide (e.g., U.S. Pat. No. 4,486,539). Detection probes not hybridized to the target polynucleotide are readily washed away from the solid support. Thus, remaining label is associated with the target polynucleotide initially present in the sample. Another technique uses a mediator polynucleotide that hybridizes to both a target polynucleotide and a polynucleotide fixed to a solid support such that the mediator polynucleotide joins the target polynucleotide to the solid support to produce a bound target (e.g., U.S. Pat. No. 4,751,177). A labeled probe can be hybridized to the bound target and unbound labeled probe can be washed away from the solid support.

By “solid support” is meant a material that is essentially insoluble under the solvent and temperature conditions of the method comprising free chemical groups available for joining an oligonucleotide or nucleic acid. Preferably, the solid support is covalently coupled to an oligonucleotide designed to bind, either directly or indirectly, a target nucleic acid. When the target nucleic acid is an mRNA, the oligonucleotide attached to the solid support is preferably a poly-T sequence. A preferred solid support is a particle, such as a micron- or submicron-sized bead or sphere. A variety of solid support materials are contemplated, such as, for example, silica, polyacrylate, polyacrylamide, metal, polystyrene, latex, nitrocellulose, polypropylene, nylon or combinations thereof. More preferably, the solid support is capable of being attracted to a location by means of a magnetic field, such as a solid support having a magnetite core. Particularly preferred supports are monodisperse magnetic spheres.

The oligonucleotide primers and probes of the present invention may be used in amplification and detection methods that use nucleic acid substrates isolated by any of a variety of well-known and established methodologies (e.g., Sambrook, J. et al., 1989, Molecular Cloning, A laboratory Manual, 2nd ed. (Cold Spring Harbor Laboratory Press, Cold Spring Harbor, N.Y.), pp. 7.37-7.57; Lin, L. et al., 1993, “Simple and Rapid Sample Preparation Methods for Whole Blood and Blood Plasma” in Diagnostic Molecular Microbiology, Principles and Applications (Persing, D. H. et al., Eds., American Society for Microbiology, Washington, D.C.), pp. 605-616); Ausubel et al., Current Protocols in Molecular Biology, John Wiley & Sons, New York (2001 and later updates thereto). In one illustrative example, the target mRNA may be prepared by the following procedure to yield mRNA suitable for use in amplification. Briefly, cells in a biological sample (e.g., peripheral blood or bone marrow cells) are lysed by contacting the cell suspension with a lysing solution containing at least about 150 mM of a soluble salt, such as lithium halide, a chelating agent and a non-ionic detergent in an effective amount to lyse the cellular cytoplasmic membrane without causing substantial release of nuclear DNA or RNA. The cell suspension and lysing solution are mixed at a ratio of about 1:1 to 1:3. The detergent concentration in the lysing solution is between about 0.5-1.5% (v/v). Any of a variety of known non-ionic detergents are effective in the lysing solution (e.g., TRITON®-type, TWEEN®-type and NP-type); typically, the lysing solution contains an octylphenoxy polyethoxyethanol detergent, preferably 1% TRITON® X-102. This procedure may work advantageously with biological samples that contain cell suspensions (e.g., blood and bone marrow), but it works equally well on other tissues if the cells are separated using standard mincing, screening and/or proteolysis methods to separate cells individually or into small clumps. After cell lysis, the released total RNA is stable and may be stored at room temperature for at least 2 hours without significant RNA degradation without additional RNase inhibitors. Total RNA may be used in amplification without further purification or mRNA may be isolated using standard methods generally dependent on affinity binding to the poly-A portion of mRNA.

In certain embodiments, mRNA isolation employs capture particles consisting essentially of poly-dT oligonucleotides attached to insoluble particles. The capture particles are added to the above-described lysis mixture, the poly-dT moieties annealed to the poly-A mRNA, and the particles separated physically from the mixture. Generally, superparamagnetic particles may be used and separated by applying a magnetic field to the outside of the container. Preferably, a suspension of about 300 pg of particles (in a standard phosphate buffered saline (PBS), pH 7.4, of 140 mM NaCl) having either dT₁₄ or dT₃₀ linked at a density of about 1 to 100 pmoles per mg (preferably 10-100 pmols/mg, more preferably 10-50 pmols/mg) are added to about 1 mL of lysis mixture. Any superparamagnetic particles may be used, although typically the particles are a magnetite core coated with latex or silica (e.g., commercially available from Serodyn or Dynal) to which poly-dT oligonucleotides are attached using standard procedures (Lund et al., Nuc. Acids Res., 1988, 16:10861-10880). The lysis mixture containing the particles is gently mixed and incubated at about 22-42° C. for about 30 minutes, when a magnetic field is applied to the outside of the tube to separate the particles with attached mRNA from the mixture and the supernatant is removed. The particles are washed one or more times, generally three, using standard resuspension methods and magnetic separation as described above. Then, the particles are suspended in a buffer solution and can be used immediately in amplification or stored frozen.

A number of parameters may be varied without substantially affecting the sample preparation. For example, the number of particle washing steps may be varied or the particles may be separated from the supernatant by other means (e.g., filtration, precipitation, centrifugation). The solid support may have nucleic acid capture probes affixed thereto that are complementary to the specific target sequence or any particle or solid support that non-specifically binds the target nucleic acid may be used (e.g., polycationic supports as described, for example, in U.S. Pat. No. 5,599,667). For amplification, the isolated RNA is released from the capture particles using a standard low salt elution process or amplified while retained on the particles by using primers that bind to regions of the RNA not involved in base pairing with the poly-dT or in other interactions with the solid-phase matrix. The exact volumes and proportions described above are not critical and may be varied so long as significant release of nuclear material does not occur. Vortex mixing is preferred for small-scale preparations but other mixing procedures may be substituted. It is important, however, that samples derived from biological tissue be treated to prevent coagulation and that the ionic strength of the lysing solution be at least about 150 mM, preferably 150 mM to 1 M, because lower ionic strengths lead to nuclear material contamination (e.g., DNA) that increases viscosity and may interfere with amplification and/or detection steps to produce false positives. Lithium salts are preferred in the lysing solution to prevent RNA degradation, although other soluble salts (e.g., NaCl) combined with one or more known RNase inhibitors would be equally effective.

The above descriptions are intended to be exemplary only. It will be recognized that numerous other assays exist that can be used for amplifying and/or detecting mRNA expression in biological samples. Such methods are also considered within the scope of the present invention.

A variety of protocols for detecting and/or measuring the level of expression of polypeptides, using either polyclonal or monoclonal antibodies specific for the product, are known in the art. Examples include enzyme-linked immunosorbent assay (ELISA), immunohistochemistry (IHC), radioimmunoassay (RIA), fluorescence activated cell sorting (FACS), and the like. A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.); Maddox, D. E. et al. (1983; J. Exp. Med. 158:1211-1216); Harlow and Lane (Antibodies: A Laboratory Manual, Cold Spring Harbor Laboratory, 1988); and Ausubel et al., Current Protocols in Molecular Biology, John Wiley & Sons, New York (2001 and later updates thereto).

In general, the presence or absence of a cancer in a patient may be determined by (a) contacting a biological sample obtained from a patient with binding agents specific for one or more of the cancer-associated markers selected from the group consisting of BL1792, BL1794, BL1798, BL1799, BL1801, and BL1811; (b) detecting in the sample a level of polypeptide that binds to each binding agent; and, (c) comparing the level of polypeptide with a predetermined cut-off value, wherein a level of polypeptide present in a biological sample that is above the predetermined cut-off value for one or more marker is indicative of the presence of cancer cells in the biological sample.

In one illustrative embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length proteins and polypeptide portions thereof to which the binding agent binds, as described above.

The solid support may be any material known to those of ordinary skill in the art to which the protein may be attached. For example, the solid support may be a test well in a microtiter plate or a nitrocellulose or other suitable membrane. Alternatively, the support may be a bead or disc, such as glass, fiberglass, latex, or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment, which may be a direct linkage between the agent and functional groups on the support or may be a linkage by way of a cross-linking agent. Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 pg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at Al2-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample and polypeptide is allowed to bind to the antibody. The sample may be diluted with a suitable diluent, such as phosphate-buffered saline (PBS), prior to incubation. In general, an appropriate contact time (i.e., incubation time) is a period of time that is sufficient to detect the presence of polypeptide within a sample obtained from an individual with cancer. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above as well as other known in the art.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as bladder cancer, the signal detected from the reporter group that remains bound to the solid support is generally compared to a signal that corresponds to a predetermined cut-off value. In one embodiment, the cut-off value for the detection of a cancer is the average mean signal obtained when the immobilized antibody is incubated with samples from patients without the cancer. In another embodiment, a sample generating a signal that is three standard deviations above the predetermined cut-off value is considered positive for the cancer. In another embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. In certain embodiments, the amount of antibody immobilized on the membrane ranges from about 25 ng to about 1 μg, and in other embodiments is from about 50 ng to about 500 ng. Such tests can typically be performed with a very small amount of biological sample.

In other embodiments of the invention, the cancer-associated polypeptides described herein may be utilized to detect the presence of antibodies specific for the polypeptides in a biological sample. The detection of such antibodies specific for cancer-associated polypeptides may be indicative of the presence of cancer in the patient from which the biological sample was derived. In one illustrative example, a biological sample is contacted with a solid phase to which one or more cancer-associated polypeptides, such as recombinant or synthetic BL1792, BL1794, BL1798, BL1799, BL1801, or BL1811 polypeptides, or portions thereof, have been attached. In certain other embodiments, the cancer-associated polypeptides used in this aspect of the invention comprise one or more polypeptides, or portions thereof, selected from the group consisting of BL1792, BL1794, BL1798, BL1799, BL1801, and BL1811. In a further embodiment, the cancer-associated polypeptides used in this aspect of the invention comprise two or more polypeptides, or portions thereof, selected from the group consisting of BL1792, BL1794, BL1798, BL1799, BL1801, and BL1811. In one illustrative embodiment, the biological sample tested according to this aspect of the invention is a peripheral blood sample. A biological sample is generally contacted with the polypeptides for a time and under conditions sufficient to form detectable antigen/antibody complexes. Indicator reagents may be used to facilitate detection, depending upon the assay system chosen. In another embodiment, a biological sample is contacted with a solid phase to which a recombinant or synthetic polypeptide is attached and is also contacted with a monoclonal or polyclonal antibody specific for the polypeptide, which preferably has been labeled with an indicator reagent. After incubation for a time and under conditions sufficient for antibody/antigen complexes to form, the solid phase is separated from the free phase and the label is detected in either the solid or free phase as an indication of the presence of antibodies. Other assay formats utilizing recombinant and/or synthetic polypeptides for the detection of antibodies are available in the art and may be employed in the practice of the present invention.

The above descriptions are intended to be exemplary only. It will be recognized that numerous other assays exist that can be used for detecting polypeptide expression in the methods of the present invention. Such methods are considered within the scope of the present invention. Unless mentioned otherwise, the techniques employed or contemplated herein are standard methodologies well-known to one of ordinary skill in the art. The examples of embodiments that follow are provided for illustration only.

### EXAMPLE 1

### Identification of cDNAs encoding bladder tumor proteins From a PCR-Based Subtraction Library

This Example illustrates the identification of cDNA molecules encoding bladder tumor proteins.

A PCR based subtraction method, as developed by Clontech (Palo Alto, Calif.), was utilized to create a library (BLAM01) enriched for bladder tumor-specific antigens. Transcripts from a pool of three bladder tumors (Tester) were subtracted with a set of transcripts from normal tissues (Driver) consisting of brain, pancreas, bone marrow, lung, skeletal muscle, heart, kidney, liver, small intestine, and bladder. The subtraction was executed according to the manufacturer's recommendations with the following changes: 1) cDNA was restricted with a mixture of enzymes, including Mscl, PvuII, Stul, and DraI, instead of the Clontech recommended single enzyme, Rsal; 2) The ratio of driver to tester cDNA was increased to 76:1 in the hybridization steps to give a more stringent subtraction. Following the PCR amplification steps, the cDNAs were cloned into the pCR2.1-TOPO (Invitrogen) plasmid vector. To analyze the efficiency of the subtraction, the housekeeping gene, actin, was PCR amplified from dilutions of subtracted as well as unsubtracted PCR samples. Furthermore, the complexity and redundancy of the library was characterized by screening 96 clones. Of these, 85 had inserts that could be sequenced and analyzed by comparing to available databases (Genbank, GenSeq, huEST). These analyses suggest that the library is enriched for genes overexpressed in bladder tumor samples.

An additional 3,360 clones were screened yielding 2,772 sequences.

### EXAMPLE 2

### Analysis of mRNA Expression Using Microarray Technology

This Example describes the characterization of bladder tumor- and tissue-specific cDNAs/mRNAs by microarray expression analysis and subsequent nucleotide sequence analysis. Described herein are microarray expression data and nucleotide sequence for a set of elements (cDNAs) that were found to be overexpressed in bladder tumor and/or bladder normal tissue.

Using this approach, cDNA sequences were PCR amplified and their mRNA expression profiles in tumor and normal tissues were examined using cDNA microarray technology essentially as described (Shena, M. et al., 1995 Science 270:467-70). In brief, the clones were arrayed onto glass slides as multiple replicas, with each location corresponding to a unique cDNA clone (as many as 5500 clones can be arrayed on a single slide, or chip). Each chip was hybridized with a pair of cDNA probes that are fluorescence-labeled with Cy3 and Cy5, respectively. Typically, 1 μg of polyA+ RNA is used to generate each cDNA probe. After hybridization, the chips were scanned and the fluorescence intensity recorded for both Cy3 and Cy5 channels. There were multiple built-in quality control steps. First, the probe quality was monitored using a panel of ubiquitously expressed genes. Secondly, the control plate also included yeast DNA fragments of which complementary RNA may be spiked into the probe synthesis for measuring the quality of the probe and the sensitivity of the analysis. Currently, the technology offers a sensitivity of 1 in 100,000 copies of mRNA. Finally, the reproducibility of this technology was ensured by including duplicated control cDNA elements at different locations.

The clones analyzed on the chip were part of a multi-tumor chip analysis and were randomly picked clones from a bladder tumor PCR subtraction library (BLAM01; see Example 1), a thyroid tumor PCR subtracted library, and a consolidated liver tumor PCR subtracted library. A total of 5410 clones (1056 from the BLAM01 library) were arrayed.

cDNA inserts for arraying were amplified by PCR using vector specific primers. The arrays were probed with 33 probe pairs (normal tissues labeled with Cy5 and tumor-specific probes labeled with Cy3). Computational analysis was then performed which consisted of determining the ratio of the mean hybridization signal for a particular element (cDNA) using two sets of probes. The ratio is a reflection of the over- or under-expression of the element (cDNA) within a probe population. Probe groups were set up to identify cDNAs with high differential expression in probe group#1 as compared to probe group#2. Probe group#1 consisted of 3 bladder tumor probes (each a pool of 2 independent bladder tumor samples), whereas probe group#2 consisted of 33 normal tissues, including normal bladder tissue. A threshold (fold-overexpression in probe group#1) was set at 2.0. This threshold was set to identify elements with overexpression that could be reproducibly detected based on the quality of the chip.

Certain elements identified by the above analysis are shown in Table 2. Elements were grouped according to their value for mean signal 1 (probe group#1; bladder tumor) and mean signal 2 (probe group#2; normal tissues including normal bladder) and ranked by ratio (threshold of overexpression). Elements which had the potential for no normal tissue expression (mean 2<0.1) were grouped in “high” (mean 1>2.0), “medium” (mean 1=0.1-0.2) and “low” (mean 1<0.1) expression groups. Elements that had a mean 2 of 0.1-0.2 were considered as having a potential for low normal tissue expression and elements that had a mean signal 2>0.2 were considered as candidates with a potential for higher normal tissue expression. Based on this initial analysis and visual analysis of the associated MicroArray data, elements with “high” (mean 1>2.0), “medium” (mean 10.1-0.2) and a “potential for low normal expression” (mean 2 of 0.1-0.2) were considered further as being candidates exhibiting overexpression in bladder tumor (and potentially normal bladder tissue). These elements were sequenced, and used as a query in a Blast search of public databases in order to confirm/deduce their identity. cDNAs are shown in the table as having some degree of similarity with known sequences or with sequences with unknown function. Twenty-two candidates were identified in this analysis. All of the 22 identified candidates originated from the bladder library (BLAM01) even though only 20% (1056/5410) of the elements on the chip were from this library. No other candidates previously identified as having overexpression associated with other non-bladder malignancies (n=172), were identified by this analysis, indicating that the analysis identified genes associated with overexpression specific to bladder tumor and normal bladder tissue and not to other tissue types (tumor or normal). This observation further validates the microarray analysis. Of these 22 candidates, 6 were of particular interest and are shown in Table 2.

TABLE 2
SUMMARY OF MICROARRAY AND SEQUENCE ANALYSIS OF cDNA MOLECULES ENCODING BLADDER TUMOR PROTEINS

| | | | | Mean | Mean | | | | |
|---|---|---|---|---|---|---|---|---|---|
| SEQ | Element | Element | | Signal | Signal | Corixa | | GenBank | |
| ID NO | (384) | (96) | Ratio | 1 | 2 | Plate # | Candidate | ID | Description |
| 1-3 | PCX368:r01c04 | 901:B2 | 19.99 | 1.831 | 0.092 | BLAM26 B2 | BL1792P | 11275674 | Short-chain alcohol |
| | | | | | | | | | dehydrogenase (HEP27) |
| 4-6 | PCX368:r02c06 | 901:D3 | 3.64 | 0.404 | 0.111 | BLAM26 D3 | BL1794P | 914900 | GAGE-2 protein |
| 7-9 | PCX368:r06c14 | 902:D7 | 11.22 | 1.066 | 0.095 | BLAM27 D7 | BL1798P | 1621394 | Interleukin-13 receptor |
| 10-14 | PCX370:r10c05 | 911:C3 | | | | BLAM25 C3 | BL1799P* | 190725 | cDNA for parathyroid hormone- |
| | | | | | | | | | related protein |
| 15-17 | PCX370:r06c01 | 910:C1 | 6.42 | 0.912 | 0.142 | BLAM35 C1 | BL1801P | 3603252 | Keratin 13 |
| 18 | PCX369:r05c19 | 906:A10 | 8.2 | 0.447 | 0.054 | BLAM31 A10 | BL1811P | 8748931 | gDNA from 7 p 11.2-p 21, (clone |
| | | | | | | | | | RP11-507K12) |

*This candidate was also identified as being overexpressed in lung tumors. In particular, it was shown to be overexpressed in all lung squamous tumors, and expression in normal breast was also observed by microarray analyses. Real-time RT-PCR analyses has confirmed the microarray data.

Several candidates were identified using the above analysis and were designated BL1792P, BL1794, BL1798, BL1799, BL1801, and BL1811. Nucleotide sequences for the elements with “high” (mean 1>2.0), “medium” (mean 10.1-0.2) and a “potential for low normal expression” (mean 2 of 0.1-0.2), are disclosed herein as SEQ ID NOs:1-2, 4-5, 7-8, 10-12, 15-16 and 18. Where available, “full length” nucleotide sequence and ORFs are presented (designated as FL and ORF, respectively, in the section entitled “Brief Description of Sequence Identifiers”). The amino acid sequences are provided in SEQ ID NOs:3, 6, 9, 13-14 and 17 and are also detailed in the section entitled “Brief Description of Sequence Identifiers”.

In summary, the sequences identified herein are overexpressed in bladder tumors and represent cancer-associated markers for bladder cancer. Accordingly, these sequences have utility as diagnostic markers for the detection and monitoring of bladder malignancy.

### EXAMPLE 3

### Analysis of cDNA Expression Using Real-Time PCR

Real-time PCR (see Gibson et al., Genome Research 6:995-1001, 1996; Heid et al., Genome Research 6:986-994, 1996) is a technique that evaluates the level of PCR product accumulation during amplification. This technique permits quantitative evaluation of mRNA levels in multiple samples. Briefly, mRNA is extracted from tumor and normal tissue and cDNA is prepared using standard techniques. Real-time PCR was used to further evaluate the expression patterns of the cancer-associated markers identified in Examples 1 and 2.

Real-time PCR was performed using a Perkin Elmer/Applied Biosystems (Foster City, Calif.) 7700 Prism instrument. Matching primers and fluorescent probes were designed for the genes of interest using the primer express program provided by Perkin Elmer/Applied Biosystems (Foster City, Calif.). Optimal concentrations of primers and probes were initially determined and control (e.g., β-actin) primers and probes were obtained commercially from Perkin Elmer/Applied Biosystems (Foster City, Calif.). To quantitate the amount of specific RNA in a sample, a standard curve was generated using a plasmid containing the gene of interest. Standard curves were generated using the Ct values determined in the real-time PCR, which are related to the initial cDNA concentration used in the assay. Standard dilutions ranging from 10-10⁶ copies of the gene of interest are generally sufficient. In addition, a standard curve was generated for the control sequence. This permits standardization of initial RNA content of a tissue sample to the amount of control for comparison purposes.

An alternative real-time PCR procedure can be carried out as follows: The first-strand cDNA to be used in the quantitative real-time PCR is synthesized from 20 μg of total RNA that is first treated with DNase I (e.g., Amplification Grade, Gibco BRL Life Technology, Gaitherburg, Md.), using Superscript Reverse Transcriptase (RT) (e.g., Gibco BRL Life Technology, Gaitherburg, Md.). Real-time PCR is performed, for example, with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence is monitored during the whole amplification process. The optimal concentration of primers is determined using a checkerboard approach and a pool of cDNAs from bladder tumors is used in this process. The PCR reaction is performed in 25 μl volumes that include 2.5 μl of SYBR green buffer, 2 μl of cDNA template and 2.5 μl each of the forward and reverse primers for the gene of interest. The cDNAs used for RT reactions are diluted approximately 1:10 for each gene of interest and 1:100 for the β-actin control. In order to quantitate the amount of specific cDNA (and hence initial mRNA) in the sample, a standard curve is generated for each run using the plasmid DNA containing the gene of interest. Standard curves are generated using the Ct values determined in the real-time PCR which are related to the initial cDNA concentration used in the assay. Standard dilution ranging from 20−2×10⁶ copies of the gene of interest are used for this purpose. In addition, a standard curve is generated for β-actin ranging from 200 fg-2000 fg. This enables standardization of the initial RNA content of a tissue sample to the amount of β-actin for comparison purposes. The mean copy number for each group of tissues tested is normalized to a constant amount of β-actin, allowing the evaluation of the over-expression levels seen with each of the genes.

Real-time analysis showed that BL1792P is overexpressed by at least 4-fold in 4 of 7 bladder tumors as compared to all normal tissues tested, including normal bladder. BL1792P was also overexpressed by at least 2-fold in two of two liver tumor samples tested as compared to all normal tissues tested, including normal liver.

BL1798P was overexpressed in nearly all bladder tumors tested as compared to normal bladder. Very high overexpression was observed in one particular bladder tumor as compared to all normal tissues tested. BL1798P expression was observed in normal kidney, liver, thyroid, prostate, heart, spinal cord and lymph node. Lower expression was observed in normal skeletal muscle, intestine, lung, pancreas, brain, and breast.

BL1799P was overexpressed in 3 of 7 bladder tumors as compared to most normal tissues, including normal bladder. Expression of this marker was observed in normal prostate, brain, and breast.

Overexpression of BL1801P was observed in 3 of 7 bladder tumors as compared to most normal tissues including normal bladder. Expression was also observed in normal esophagus and lymph node.

Overexpression of BL1811P was observed in 3 of 7 bladder tumors and 2 of 2 kidney tumors as compared to normal tissues tested including normal bladder and normal kidney. Low expression was observed in lymph node.

Thus, real-time PCR expression analysis confirms the microarray analysis and shows that BL1792P, BL1794P, BL1798P, 8L1799P, BL1801P, and BL1811P are bladder cancer-associated markers that can be used for the detection and diagnosis of bladder malignancies. Additionally, BL1792P was found to be overexpressed in liver tumors and thus can be used as a liver cancer marker. Similarly, BL1811P was shown to be overexpressed in kidney tumors and thus can be used as a kidney cancer marker.

### EXAMPLE 4

### Generation and Characterization of Monoclonal Antibodies Specific for Cancer-Assoclated Polypeptides

Mouse monoclonal antibodies are raised against E. coli derived cancer-associated proteins as follows: Mice are immunized with Complete Freund's Adjuvant (CFA) containing 50 μg recombinant tumor protein, followed by a subsequent intraperitoneal boost with Incomplete Freund's Adjuvant (IFA) containing 10 μg recombinant protein. Three days prior to removal of the spleens, the mice are immunized intravenously with approximately 50 μg of soluble recombinant protein. The spleen of a mouse with a positive titer to the tumor antigen is removed, and a single-cell suspension made and used for fusion to SP2/O myeloma cells to generate B cell hybridomas. The supernatants from the hybrid clones are tested by ELISA for specificity to recombinant tumor protein, and epitope mapped using peptides that spanned the entire tumor protein sequence. The mAbs are also tested by flow cytometry for their ability to detect tumor protein on the surface of cells stably transfected with the cDNA encoding the tumor protein.

### EXAMPLE 5

### Synthesis of Polypeptides

Polypeptides are synthesized on a Perkin Elmer/Applied Biosystems Division 430A peptide synthesizer using FMOC chemistry with HPTU (O-Benzotriazole-N,N,N′,N′-tetramethyluronium hexafluorophosphate) activation. A Gly-Cys-Gly sequence is attached to the amino terminus of the peptide to provide a method of conjugation, binding to an immobilized surface, or labeling of the peptide. Cleavage of the peptides from the solid support is carried out using the following cleavage mixture: trifluoroacetic acid:ethanedithiol:thioanisole:water:phenol (40:1:2:2:3). After cleaving for 2 hours, the peptides are precipitated in cold methyl-t-butyl-ether. The peptide pellets are then dissolved in water containing 0.1% trifluoroacetic acid (TFA) and lyophilized prior to purification by C18 reverse phase HPLC. A gradient of 0%-60% acetonitrile (containing 0.1% TFA) in water (containing 0.1% TFA) is used to elute the peptides. Following lyophilization of the pure fractions, the peptides are characterized using electrospray or other types of mass spectrometry and by amino acid analysis.

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A composition for detecting bladder cancer cells in a biological sample comprising an oligonucleotide specific for any one of the cancer-associated polynucleotides recited in SEQ ID NOs: 1-2, 4-5, 7-8, 10-12, 15-16 and 18, or the complement thereof.

2. A composition for detecting bladder cancer cells in a biological sample comprising at least two oligonucleotide primers specific for any one of the cancer-associated polynucleotides recited in SEQ ID NOs: 1-2, 4-5, 7-8, 10-12, 15-16 and 18, or the complement thereof.

3. A composition for detecting bladder cancer cells in a biological sample comprising at least two of:
a) a first oligonucleotide primer pair specific for any one of the polynucleotides recited in SEQ ID NOs: 1-2, 4-5, 7-8, 10-12, 15-16 and 18, or the complement thereof;
b) a second oligonucleotide primer pair specific for any one of the polynucleotides recited in SEQ ID NOs: 1-2, 4-5, 7-8, 10-12, 15-16 and 18, or the complement thereof;
c) a third oligonucleotide primer pair specific for any one of the polynucleotides recited in SEQ ID NOs: 1-2, 4-5, 7-8, 10-12, 15-16 and 18, or the complement thereof; and
d) a fourth oligonucleotide primer pair specific for any one of the polynucleotides recited in SEQ ID NOs: 1-2, 4-5, 7-8, 10-12, 15-16 and 18, or the complement thereof;
e) a fifth oligonucleotide primer pair specific for any one of the polynucleotides recited in SEQ ID NOs: 1-2, 4-5, 7-8, 10-12, 15-16 and 18, or the complement thereof;
f) a sixth oligonucleotide primer pair specific for any one of the polynucleotides recited in SEQ ID NOs: 1-2, 4-5, 7-8, 10-12, 15-16 and 18, or the complement thereof;
wherein the first, second, third, fourth, fifth, and sixth primer pairs are specific for different polynucleotides from among the polynucleotides recited in SEQ ID NOs: 1-2, 4-5, 7-8, 10-12, 15-16 and 18, or the complement thereof.

4. A composition for detecting bladder cancer cells in a biological sample comprising any one or more of the polypeptide sequences recited in SEQ ID NOs: 3, 6, 9, 13-14 and 17 or a polypeptide sequence encoded by a polynucleotide sequence set forth in any one of SEQ ID NOs: 1-2, 4-5, 7-8, 10-12, 15-16 and 18, or a fragment of any of said polypeptide wherein said fragment is useful in the detection of bladder cancer cells.

5. A composition for detecting bladder cancer cells in a biological sample comprising an antibody that specifically recognizes any one of the polypeptide sequences recited in SEQ ID NOs: 3, 6, 9, 13-14 and 17 or a polypeptide sequence encoded by a polynucleotide sequence set forth in any one of SEQ ID NOs: 1-2, 4-5, 7-8, 10-12, 15-16 and 18.

6. A diagnostic kit for detecting bladder cancer cells in a biological sample comprising the composition according to claim 1.

7. A diagnostic kit for detecting bladder cancer cells in a biological sample comprising the composition according to claim 2.

8. A diagnostic kit for detecting bladder cancer cells in a biological sample comprising the composition according to claim 3.

9. A diagnostic kit for detecting antibodies specific for a cancer-associated marker in a biological sample comprising the composition according to claim 4.

10. A diagnostic kit for detecting bladder cancer cells in a biological sample comprising the composition according to claim 5.

11.-16. (canceled)

17. A method for detecting the presence of bladder cancer cells in a biological sample comprising the steps of:
(a) detecting the level of expression in the biological sample of any one or more of the cancer-associated markers selected from the group consisting of BL1792, BL1794, BL1798, BL1799, BL1801, and BL1811; and
(b) comparing the level of expression detected in the biological sample for each marker to a predetermined cut-off value for each marker;
wherein a detected level of expression above the predetermined cut-off value for one or more markers is indicative of the presence of cancer cells in the biological sample.

18. The method of claim 17, wherein step (a) comprises detecting the level of mRNA expression.

19. The method of claim 18, wherein step (a) comprises detecting the level of mRNA expression using a nucleic acid hybridization technique.

20. The method of claim 18, wherein step (a) comprises detecting the level of mRNA expression using a nucleic acid amplification method.

21. The method of claim 20, wherein step (a) comprises detecting the level of mRNA expression using a nucleic acid amplification method selected from the group consisting of transcription-mediated amplification (TMA), polymerase chain reaction amplification (PCR), reverse-transcription polymerase chain reaction amplification (RT-PCR), ligase chain reaction amplification (LCR), strand displacement amplification (SDA), and nucleic acid sequence based amplification (NASBA).

22. The method of claim 18, wherein the cancer-associated marker comprises a nucleic acid sequence set forth in any one of SEQ ID NOs: 1-2, 4-5, 7-8, 10-12, 15-16 and 18 or a nucleic acid sequence encoding an amino acid sequence set forth in any one of SEQ ID NOs: 3, 6, 9, 13-14 and 17.

23. The method of claim 17, wherein step (a) comprises detecting the level of protein expression.

24. The method of claim 23, wherein step (a) comprises detecting the level of protein expression using an immunoassay.

25. The method of claim 24, wherein step (a) comprises detecting the level of protein expression using an immunoassay selected from the group consisting of an ELISA, an immunohistochemical assay, an immunocytochemical assay, and a flow cytometry assay of antibody-labeled cells.

26. The method of claim 23, wherein the cancer-associated marker comprises an amino acid sequence set forth in any one of SEQ ID NOs: 3, 6, 9, 13-14 and 17 or a polypeptide sequence encoded by a polynucleotide sequence set forth in any one of SEQ ID NOs: 1-2, 4-5, 7-8, 10-12, 15-16 and 18.

27. The method of claim 17, wherein the biological sample is a sample suspected of containing cancer-associated markers, antibodies to such cancer-associated markers or cancer cells expressing such markers or antibodies.

28. The method of claim 27, wherein the biological sample is selected from the group consisting of a biopsy sample, lavage sample, sputum sample, serum sample, peripheral blood sample, lymph node sample, bone marrow sample, urine sample, and pleural effusion sample.
